(19) United States
(12) Patent Application Publication
(10) Pub. No.: US 2020/0289137 A1
(43) Pub. Date: Sep. 17, 2020

(54) Machine for slowing the flow of time and extending life

(71) Applicant: Paul Alex LaViolette, Niskayuna, NY (US)

(72) Inventor: Paul Alex LaViolette, Niskayuna, NY (US)

(21) Appl. No.: 16/602,550

(22) Filed: Oct. 30, 2019

Related U.S. Application Data
(60) Provisional application No. 62/751,795, filed on Oct. 29, 2018.

(51) Int. Cl. A61B 17/22

(52) U.S. Cl. CPC A61B 17/22022; B64G 9/00

(57) ABSTRACT

Scalar-longitudinal waves of a particular type are disclosed here which have the ability to slow down clock-measured time flow as well as the rate of all physical processes in a manner similar to the phenomenon of relativistic time dilation, but where said slowing occurs in a stationary frame of reference. An apparatus consisting of a high-voltage DC power supply whose high-voltage output is discharged through a thyratron to a dome electrode to produce a repeating series of scalar-longitudinal DC shock waves of short rise-time and arranged to pass through a target object or person for the purpose of slowing down the rate of flow of time for said target object or person.

### REFERENCES CITED

### U.S. Patents

U.S. Pat. No. 9,306,527 B1 April 2016 Hively

### U.S. Applications

Ser. No. 62/751,795 Oct. 29, 2018 LaViolette

### OTHER REFERENCES

Erlichson, H. “The rod contraction-clock retardation ether theory and the special theory of relativity,” Amer. J. Phys. 41 (1973): 1068-1077.
Gasser, W. G. “Experimental clarification of Coulomb-field propagation: Superluminal information transfer confirmed by simple experiment.” (2016). Available at: http://www.pandualism.com/c/coulomb experiment. html
Gillabel, D. “The Bee Machine or Teslatron,” paper posted on the website: www.soul-guidance.com/houseofthesun/teslatron.html.
Hively, L. M. and Loebl, A. S. “Classical and extended electrodynamics. Physics Essays 32(1) (2019): 112-126.
LaViolette, P. A. “An introduction to subquantum kinetics: Part I” Intl. J. General Systems 11 (1985a): 281-294.
LaViolette, P. A. “An introduction to subquantum kinetics: Part II” Intl. J. General Systems 11 (1985b): 295-328.
LaViolette, P. A. “The electric charge and magnetization distribution of the nucleon: Evidence of a subatomic Turing wave pattern.” Intl. J. General Systems 37(6) (2008a): 649-676; eprint at: starburstfound.org/downloads/physics/nucleon.pdf
LaViolette, P. A. Secrets of Antigravity Propulsion. Bear & Co., Rochester, Vt., 2008b; Ch. 6, Sec. 6.2.
LaViolette, P. A. “The cosmic ether: Introduction to subquantum kinetics.” Space, Propulsion & Energy Sciences International Forum—2012, Physics Procedia 38 (2012a):326-349; eprint at: starburstfound.org/downloads/physics/cosmic-ether.pdf.
LaViolette, P. A. Subquantum Kinetics. Niskayuna, N.Y.: Starlane Publications, 2012b.
LaViolette, P. A. “A method for slowing the flow of time.” Oct. 30, 2018, posted at: etheric.com/slowing-time-flow/.
Mendeleev, D. “An attempt towards a chemical conception of the ether.” Imperial Mint, St. Petersburg, Longmans, Green & Co, NY (1904); eprint at: bourabai.kz/mendeleev/ether.html.
Reed, D. “Unraveling the potentials puzzle and corresponding case for the scalar longitudinal electrodynamic wave.” J. Phys.: Conf. Ser. 1251 (2019) 012043; eprint at: researcgate.net/publication/333977851 Unraveling_the_potential_spuzzle_and_corresponding_case_for_the_scalar_longitudinal_electrodynamic_wave.
Vassilatos, G. Secrets of Cold War Technology. Borderland Sciences, Baside, C A, 1996.

### FIELD OF THE DISCLOSURE

This disclosure relates to systems, apparatuses, and methods for generating and/or utilizing scalar-longitudinal shock waves for the purpose of slowing down the flow of time in a local frame of reference.

### DESCRIPTION OF PRIOR ART

This application incorporates the material of provisional application 62/751,795 which the inventor filed with the USPTO on Oct. 29, 2018. It also incorporates ideas made in a website posting (etheric.com/slowing-time-flow/) made by the inventor on Oct. 30, 2018 and entitled “A method for slowing the flow of time.” Although, the discussion related in the present application modifies some of the discussion related in those prior presentations.

In recent years, NASA and other space agencies have begun to consider space expeditions to Mars and more distant planets of the Solar System. But one problem that is faced is that such flights could take many months to years to accomplish by rocket propulsion means. As a result, there has been an increased interest in methods to slow down human biological processes to prevent significant aging during such flights. This endurance problem becomes more severe when considering interstellar flights to nearby star systems, a prospect which has received increased attention since the discovery of habitable planets in our local stellar environs, a few lying within 6 light years of the Sun. But passenger endurance of such flights presents an even more formidable obstacle since the flights could take decades to accomplish.

One solution would be to propel the spaceship to a velocity close to the speed of light where relativistic time dilation would act to reduce the rate of passenger aging. For example, a journey to Alpha Centauri, which lies about 4.3 light years away, would last almost 5 years if the spaceship were accelerated to 0.9c, 90% of the speed of light. However, according to special relativity, the journey for the occupants the journey would last only about two years due to the effects of relativistic time dilation. So, even if they were to travel at near light speed velocities to nearby stars, passengers would be required to endure a passage of many years.

One approach to the problem that has been taken, and which apparently has received funding from NASA, is to place the spaceship's occupants in cryostasis, a concept often portrayed in Hollywood movies. Present investigations are aimed toward reducing a person's body temperature by a few degrees Centigrade for periods of weeks to months, a technique used during heart transplant surgeries. But this technology is in its infancy and has yet to be proven practical. One alternative solution to this problem, which is presented in the disclosure below, is to instead slow down the flow of time in the vicinity of the space traveler by electrodynamic means.

### SUMMARY

This invention relates to a shock wave-emitting apparatus that is able to slow down the flow of time in its immediate vicinity. The disclosed apparatus is capable not only of slowing down clocks, but also of slowing the rate of all physical processes occurring in the immediate vicinity of said wave-emitting apparatus. This invention also relates to a method for slowing down the flow of time in a room by exposing objects or personnel to the clock retardation effects of scalar-longitudinal DC shock waves having certain characteristics. This technology has application in space travel for slowing a traveler's rate of aging during long journeys through space to other planets in the solar system, or even to other star systems. It could also be used in the workshop or laboratory in any instance where it is beneficial to slow down time for example for technological or therapeutic purposes, or to heal or extend the life duration of a living organism. Other objects, features and advantages will become apparent as the description proceeds.

### BRIEF DESCRIPTION OF THE DRAWINGS

The foregoing and other objects and features of the invention may be better understood by reference to the following drawings:

FIG. 1A shows a succession of negative electric potential DC shock waves.

FIG. 1B shows an illustration of how the electric potential gradient of a given shock wave would induce a forward flowing ether wind.

FIG. 2 is a diagrammatic representation of a shock wave generator used to create an ether wind.

FIG. 3 is a diagrammatic representation of a shock wave generator used to create an ether wind which instead uses a charge accumulator instead of a capacitor to discharge to the thyratron.

FIG. 4 is a schematic of a clock retardation chamber.

FIG. 5 is a side view of a clock retardation chamber.

### BACKGROUND OF THE INVENTION

According to the theory of special relativity, when a clock moves relative to a given rest frame, its time retards relative to a clock that remains stationary in that rest frame, a phenomenon termed relativistic time dilation. This means that the flow of time measured by the moving clock dilates or slows down relative to that measured by the stationary clock. In other words, clocks in the moving frame are understood to proceed at a slower rate. Whenever possible we will refer to this relativistic phenomenon as “clock retardation”, rather than “time dilation”, following the thinking of Ehrlichson (1973). Clock retardation becomes most noticeable when the velocity of the clock approaches the speed of light. For example, according to standard physics, the dilated time increment t′ of the moving clock is related to the time increment t measured by the stationary clock according to the formula t′=t√/(1−v²/c²). However, the mechanism by which this clock retardation effect occurs is not explained, it is taken as a given and supported by experimental observation.

Although standard physics prefers to understand the time dilation phenomenon in the context of special relativity, the manner in which the time dilation effect is produced by the apparatus and method disclosed in the current application is best understood in the context of the ether concept. An increasing number of physicists prefer the ether concept over special relativity, considering that several experiments have shown its existence, such as the Sagnac Experiment, the Michaelson-Gale experiment, the Silvertooth experiment, and others. According to the eighteenth and nineteenth century ether theory, upon which classical electrodynamics was first developed, a moving clock would be in motion relative to the local ether frame and hence would experience an ether wind as it traveled.

One ether theory that is particularly useful for understanding this clock retardation phenomenon is the theory of subquantum kinetics (SQK); see LaViolette (1985a, b, 2008a, 2012a, b). This theory postulates an ether composed of subquantum ether units, or etherons, of various types, A, B, G, X, Y, etc. which react with one another as well as transmute one into another, and which diffuse through space. Hence it is closer to the open, reaction-diffusion system paradigm that is found in the field of chemical kinetics, rather than the closed system unitary mechanical ether paradigm of the eighteenth and nineteenth century. In many ways it is comparable to the ether conception of Mendeleev, the inventor of the periodic table. Mendeleev (1904) conceived of the ether in chemical terms as a gaseous all pervading medium consisting of at least two species, X and Y. SQK requires seven species to specify its reactions whose reactions are formalized in the reaction system called Model G. A summary of the theory is prohibitively long to be presented here and so those interested are referred to the above cited references as well as to other papers available in journals and on the internet. Over its more than 45 years of existence this theory has developed a substantial following in the scientific and engineering community.

One of the advantages of SQK is that the postulates of special and general relativity derive as corollaries of the theory; e.g., see LaViolette (2012b), Ch. 5, Sec. 5.7. In particular, SQK predicts that when a target clock is traveling through the ether, it should slow down in comparison with a stationary clock since the etheron reactions that are creating the clock and all material bodies in that vicinity are uniformly slowed down due to the effect of the relative motion of the ambient ether. That is, because an ether wind locally increases the average relative velocity of etherons in that region, the time during which etherons have a chance to encounter one another is reduced and as a result the ether reaction rate slows down. As a consequence, all physical processes and wave oscillations would slow down just as if time had slowed down.

An interesting extrapolation of this model is that the same clock retardation effect should just as well occur if the target clock were stationary in the laboratory ether reference frame, but was subject to a local ether wind of velocity v. The time dilation processes affecting the clock would be the same as though the clock were traveling through the ambient ether at velocity v. According to subquantum kinetics, such an ether wind could be artificially produced in the laboratory by electrical means.

For example, consider an apparatus that repeatedly discharges DC electrical shocks from a cathode to generate a succession of negative electric potential shock waves, or Coulomb waves. Such waves are termed scalar-longitudinal waves (SLW) since unlike Hertzian EM waves, they have little or no magnetic field component and no transverse vector potential or polarization. They are sometimes referred to as “Tesla waves” since Nikola Tesla was one of the first to experiment with these sorts of waves. They have been reported to have the ability to pass through Faraday cages unattenuated and to exhibit superluminal velocities (Hively, U.S. Pat. No. 9,306,527 B1; Hively and Loebl, 2019; Gasser, 2016; LaViolette, 2008b).

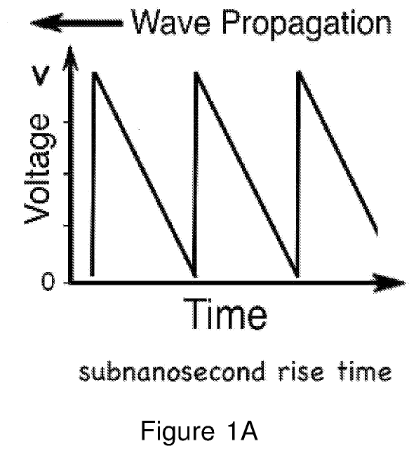

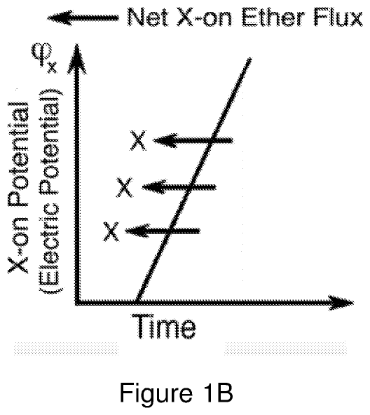

The scalar-longitudinal DC shock waves that best produce the theorized clock retardation effects disclosed here would be those having a triangular voltage profile with a steep leading-edge field gradient, each successive shock initiating with the same polarity; see FIG. 1A. In SQK, field potentials in general are represented as ether concentrations. For example, a negative electric potential, or voltage, would be represented as a positive X etheron concentration; the greater the concentration of X-etherons, the greater the magnitude of the negative voltage. The rising negative potential at the leading edge of the wave would constitute an electric field gradient that would advance forward as the scalar-longitudinal wave propagated forward. According to SQK, this scalar-longitudinal DC shock wave is modeled as a propagating X-on concentration gradient, and the steep gradient of this wave, would produce a diffusive X etheron flux directed down that gradient in the direction of wave propagation; see FIG. 1B. This flux, in turn would comprise a forward flowing X-on ether wind.

A similar manner of generating an ether wind is described in the ether theory of James Clerk Maxwell. In Maxwell's theory, which adopts Faraday's terminology, an electric field gradient produces an ether flux in the direction of the gradient's downward slope, which Faraday termed the electric flux density vector, D. In classical electrodynamics, the electric flux density varies as the gradient of the electric potential. Hence electric potential shock waves that are emitted from a cathode will induce an electric flux density vector that varies in proportion to the magnitude of the electric field gradient at the wave's leading edge. The steeper this leading edge field gradient, the greater will be the electric flux density vector. Hence D∝∇φ_{E}, where φ_{E} is the electric field potential. Subquantum kinetics adopts these same concepts. In SQK, D is equivalent to Φ_{X}, the X diffusive flux vector, whose magnitude depends on the gradient of the X etheron concentration potential; i.e., Φₓ= [symbol] _{X} ∇φ_{X}. where [symbol] _{X} is the diffusion coefficient of the x etheron specie.

Recently, an effort has been made to modify the equations of classical electrodynamics to be able to describe scalar longitudinal waves, a formulation that is called Extended Electrodynamics (EED); see Hively and Loebl (2019) and Reed (2019). The Faraday flux density vector D is referred to as the current density vector J in EED and the electric potential φ_{E} is symbolized by the scalar quantity κ. So, relation: J∝∇κ encountered in EED, serves as the equivalent of the above gradient-driven ether flux equations.

If a cathode were to emit a succession of such Coulomb potential shock waves, the wind produced by each successive shock pulse is theorized to add to the next and thereby to sustain a forward X etheron wind. In this way, it should be possible to create an X etheron wind, or “electric flux density wind”, in the laboratory by producing an apparatus that emits a succession of negative electric potential shocks having a steep leading edge gradient. Subquantum kinetics provides a more complex ether model than preceding ether theories in that it not only offers a theoretical grounding for understanding how to produce an ether wind in the laboratory by electric means, but it also offers a framework for understanding how it should be possible for such an ether wind to retard the rate of clocks or any physical process. Since in SQK the X etheron specie is critically involved in etheron reactions to produce all physical forms, matter and energy, the production of a convective X-on flux will act to slow down these etheron reactions and hence slow down all physical phenomena in the region affected, such that for an outside observer, it will appear as though time has slowed down for the affected body.

Tesla was experimenting with such electric potential DC shock waves repeating in a regular manner and used the term radiant energy to describe them. Like modern researchers, he observed that they could pass through metal shields and even attain superluminal velocities (Vassilatos, 1996). Like SQK, he understood these waves as creating longitudinal kinetic impulses in an ether. Although, by adopting the view of Mendeleev, he envisioned these ether streams as being driven by condensations and rarefactions of a gaseous ether, rather than by high and low concentrations in a reactive-diffusive ether as SQK envisions them. However, neither Tesla, nor others after him experimenting with these laboratory-produced ether winds, had discovered that such ether winds have the ability to affect the flow of time in the physical world. Hence it is maintained that this phenomenon is an original discovery of the inventor.

Based on subquantum kinetics it may be concluded that greater ether wind velocities, and hence greater time dilations, can be achieved by increasing the voltage of the shock discharge, reducing the rise-time of the shock, and increasing the pulse repetition rate of the shocks. So with proper engineering, this effect should be able to achieve degrees of time dilation suitable for application to long duration space flights.

### DETAILED DESCRIPTION

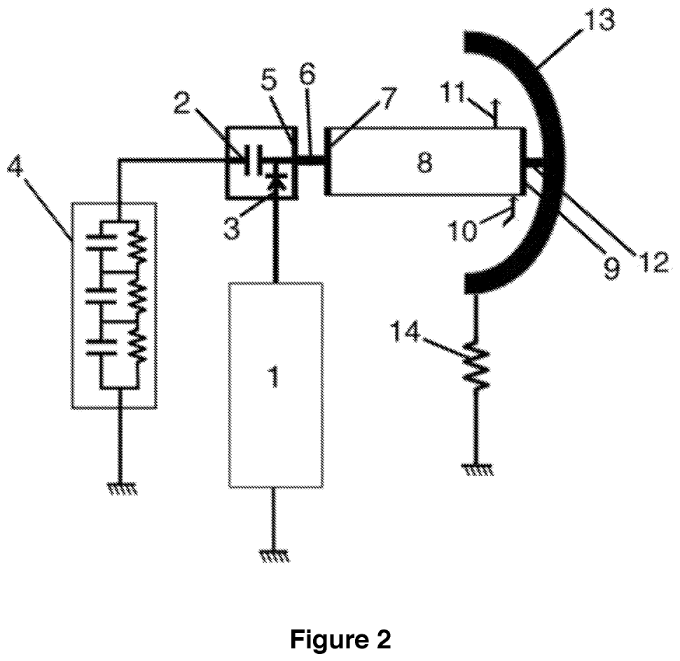

An example of a typical apparatus that could produce clock retardation in the laboratory rest frame is that shown in FIG. 2. This shows a high-voltage DC power source (1) that charges high-voltage capacitor (2) through high-voltage diode (3). Power source (1) may be either a Cockroft-Walton voltage multiplier or a high-voltage DC transformer capable of providing 250 kV or more. If a Cockroft-Walton multiplier were to be used, the low voltage side of capacitor (2) and diode (3) would be connected to the last capacitor-diode stage of the Cockroft-Walton. An oil sealed capacitor-resistor chain (4) would be needed only if power source (1) was a DC transformer. This would serve to bias capacitor (2) so that the entire voltage drop to ground does not appear across this one component. Capacitor (2) could be designed to have a capacitance of 100 pf, and together with diode (3) would be contained within an enclosure that would be filled with oil or some other suitable material that would prevent arcing. This enclosure has a metal end-plate (5) that is electrically connected in close proximity to the high-voltage end of this capacitor. Capacitor (2) is switchably connected through multi-fin spark gap thyratron (8) to dome electrode (13) via connectors (6) and (12).

In one embodiment, thyratron (8) would consist of a multi-fin spark gap having approximately 70 circular metallic fins, each fin measuring about 5 cm in diameter and 60 mils thick and fabricated from aluminum or some other noncorrosive conductive material. Adjacent fins would be separated from one another by insulating discs measuring approximately 3.7 cm in diameter and 5 mils thick made from PTFE, mica, or other suitable insulating material. Each metal fin could have a large diameter hole punched out at its center giving it a flat-ring or washer shape. This washer profile in addition could have several small-radius nubs projecting toward the fin's central axis to facilitate spark discharge in the fin's interior. Each insulating disc would similarly have a large central hole, giving it a washer-like shape. In this way, sparking would be able take place not only around the periphery of the multi-fin stack, but in its interior as well. The thyratron multi-fin stack would be contained within an enclosure ventilated by a low pressure flow of oxygen (e.g., around 3 psi). This oxygen flow would enter said thyratron enclosure through inlet port (10), be directed up the axis of the multi-fin stack to ventilate its interior space, then ventilate the periphery of the multi-fin stack, and finally exit through outlet port (11). This would serve to prevent the build up of ions which could otherwise have an undesirable effect on the proper function of the thyratron.

The outer fin at the high-voltage end of the thyratron multi-fin stack electrically connects in close proximity to metal end-plate (7) which caps the high-voltage end of thyratron (8). End plate (7), in turn, connects through conductor (6) to metal end-plate (5) which connects to capacitor (2). The outer fin at the low-voltage end of the thyratron multi-fin stack electrically connects in close proximity to metal end plate (9) which caps the low-voltage end of the thyratron. End plate (9) in turn, electrically connects to dome electrode (13) via connector (12).

With each discharge, the thyratron would fire in cascade fashion from the high-voltage end of its multi-fin stack to the low-voltage end of its multi-fin stack. In so doing, the charged released from capacitor (2) would produce a scalar-longitudinal Coulomb wave or voltage pulse that would travel forward through the thyratron's multi-fin stack, through metal end-plate (9) and electrical connector (12) to dome electrode (13). The capacitive coupling between end-plates (5) and (7) and between end-plate (9) and dome electrode (13) would assist this Coulomb wave to proceed smoothly through the thyratron to the dome electrode and outward to the space surrounding the dome with minimal inductance. The low inductance propagation of this wave is further assisted by ensuring that connectors (6) and (12) have a large diameter of at least one inch, and a short length of preferably no more than two inches. Also connection (6) leading to thyratron (8) and connection (12) leading to the center of dome (13) should be straight. These features together will ensure a path of minimum inductance between the discharge capacitor and the dome, thereby allowing a sharp pulse to be produced, having a minimal voltage rise-time. The residual charge on dome (13) is drained to ground via resistor (14) to make the dome ready to receive the next thyratron discharge. In one embodiment this resistor would have a value of 100 Mφ.

For the apparatus to effectively produce an ether wind, it is important that the multi-fin spark gap thyratron should discharge in a rhythmic manner. This will ensure that successive shock waves constructively augment one another to collectively assist in propelling the ether wind forward. This could be achieved by “tuning” the apparatus by adjusting the separation distance of the thyratron relative to discharge capacitor (2) and dome electrode (13). This could be accomplished if electrical connectors (6) and (12) were made extendible by means of concentric snugly-fitting tubes. Alternatively, rhythmic discharge could be facilitated if dome electrode (13) were to have its surface made wet by covering it with a saturated, water absorbent covering. This would create a nonlinear phase-conjugating layer on the dome's surface which could phase conjugate pulses emitted from the thyratron to radiate a time-reversed pulse back toward the thyratron to improve the regularity of its discharge. Alternatively, the thyratron discharge could be synchronized by triggering it with an external trigger circuit (not shown).

High-voltage thyratrons, other than the one described here, could also be used, one example being a hydrogen thyratron of sufficiently high-voltage capability. If a Marx bank voltage multiplier is used as the DC power source, the Marx bank multiplier would substitute for components (1) through (11), its self-pulsed output being connected directly to the center of dome (13) via connector (12).

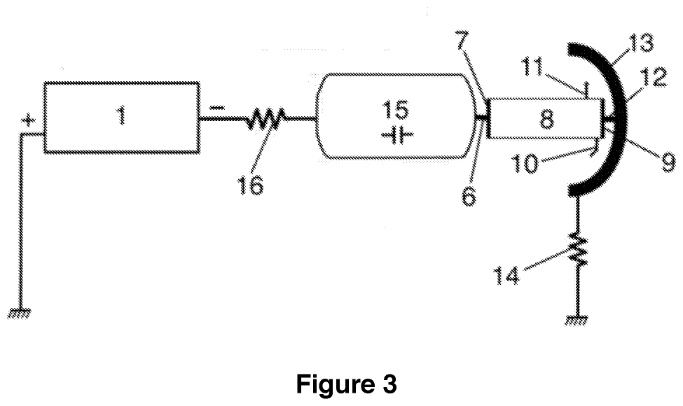

Another embodiment of this scalar-longitudinal DC shock wave apparatus is shown in FIG. 3. This is similar to the apparatus shown in FIG. 2, except that capacitor (2) and diode (3) are replaced by capacitive charge accumulator (15), which would be charged from high-voltage DC power source (1) through resistor (16). Charge accumulator (15) would then switchably connect through multi-fin spark gap thyratron (8) to dome electrode (13) via connectors (6) and (12). The charge accumulator may be designed to have a capacitance of about 100 pf and be in the form of a hollow metallic barrel-shaped electrode of diameter 104 cm and length 208 cm, or of a hollow metallic sphere of diameter 164 cm. The size of the charge accumulator could be substantially reduced by convoluting its surface. The charging resistor (16) would be designed to electrically charge the charge accumulator (15) sufficiently fast to have it achieve its maximum voltage prior to the subsequent pulse discharge. For a 250 kV DC power supply, the resistor could have a value of 60 MΩ.

In general, there are many ways that one could construct an apparatus to generate a local ether wind. The degree of time dilation produced is theorized to depend on the magnitude of the ether wind generated, which in turn is theorized to scale with the pulse voltage magnitude, abruptness of pulse discharge, and pulse repetition rate. It is preferable to use a DC power source of greater than 250 kV, a pulse rise-time of less than 800 picoseconds, and a repetition rate greater than 15 pulses per second. Increased pulse power may also have a positive influence on ether wind production. This would be determined in part by the value of capacitor (2). To attain degrees of time dilation that would be practical for long space flights, all of the above parameters would likely be scaled up from these minimal values.

Regarding the safety of this technology, scalar-longitudinal DC shock waves similar to those capable of producing clock retardation, have had a long safety record in that people have been exposed to such waves for many hours for the purpose of receiving medical therapy. In this regard, there have been documented cases of remission of cancer tumors and cures of many other diseases. So use of the device for time dilation should in addition yield beneficial effects on the human body. Some discussion of the healing abilities of scalar-longitudinal DC shock waves may be found in a paper authored by Dirk Gillabel entitled “The Bee Machine or Teslatron,” which is posted on the website: www.soul-guidance.com/houseofthesun/teslatron.html.

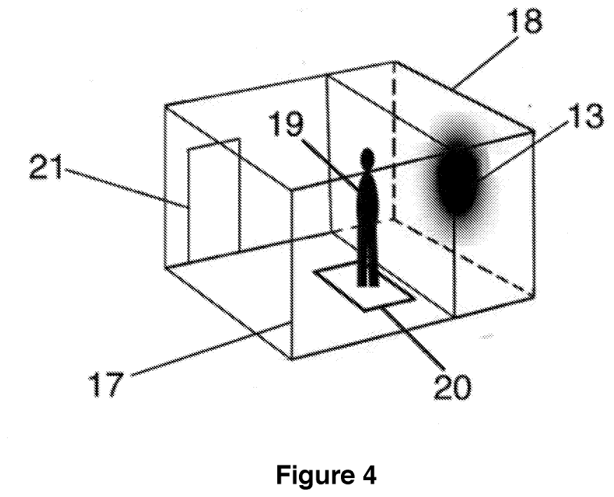

FIG. 4 is a diagram showing a chamber within which a person could undergo time dilation. The shock wave emitting cathode (13) would be located at one end of the chamber. The chamber floor, walls, and ceiling could be made of aluminum clad polyurethane panels. The rear panels (17) would be connected to ground, while the forward panels (18) would be left at a floating potential, a gap positioned about 0.3 meters in front of the dome would distance the forward from the rear panels. In this way the shocks from dome (13) would tend to be electrostatically focused in the forward direction toward the portion of the chamber where clock retardation would be experienced. The individual (19) would stand on an insulated platform (20) located within the rear portion of the chamber to experience physical time dilation. A door (21) would provide chamber access.

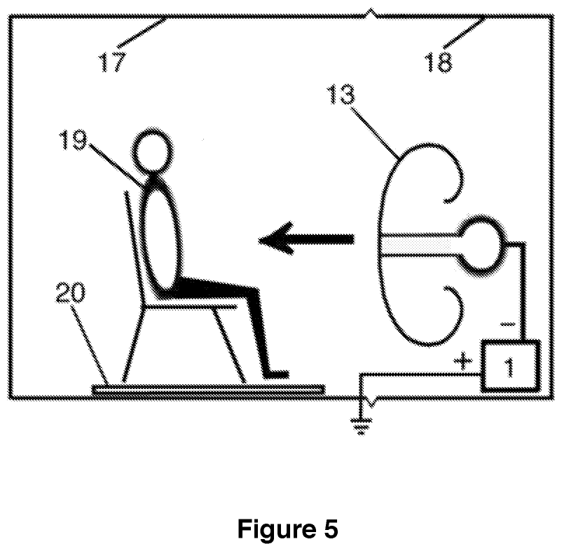

FIG. 5 shows a side-view of a time-dilation chamber. Here the subject (19) sits on an insulating platform (20) in front of dome electrode (13) which, when active, emits an ether wind indicated by the arrow. The DC power supply (1) that energizes the capacitor-thyratron component is grounded to the rear metal-clad chamber enclosure (17). The forward metal-clad chamber enclosure (18) remains at floating potential.

It is also possible to create an ether wind if the dome electrode (13) is made to emit shocks of positive, rather than negative polarity. This would be done by connecting the positive pole of the power supply to the discharge capacitor (2) and grounding the negative pole. In this case the electric flux density vector D (or X diffusive flux vector Φ_{X}) would be negative and the ether wind would blow in a reverse direction toward the dome electrode. However, presently it is not known what the health effects would be for exposure to Coulomb shock waves of positive polarity.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

What is claimed is:

1. An apparatus transmitting a repeating series of scalar-longitudinal, DC shock waves through a target object, said apparatus comprising:
a device having an electrical system that comprises:
a high-voltage DC power source electrically connected to
a capacitor that is switchably connected to
a dome electrode via
a multi-fin-spark-gap thyratron;
the electrical system configured to produce a repeating series of scalar-longitudinal DC shock waves of short rise-time; and
the device configured to direct said shock waves toward a target object, whereby the target object may experience a rate of time flow that is slowed below normal.

2. The apparatus of claim 1 wherein said capacitor, thyratron and dome electrode are together aligned in collinear fashion substantially along a central axis extending perpendicular to the dome electrode.

3. The apparatus of claim 1 wherein said capacitor is electrically connected proximate to the input end of the thyratron and where the output end of the thyratron is electrically connected proximate to the dome electrode and wherein means are provided for varying the length of said electrically connecting means.

4. The apparatus of claim 1 wherein said capacitor is connected to the input end of said thyratron by electrically connective means having a diameter sufficiently large to provide a low-inductance path for said charge being intermittently discharged through said thyratron, and where the output end of said thyratron is connected to the dome electrode by electrically connective means having a diameter sufficiently large to provide a low-inductance path for said charge being intermittently discharged to said dome electrode.

5. The apparatus of claim 1 wherein the enclosure for said thyratron is terminated at each of its ends by metal covers each electrically connected to an outer metallic fin of the thyratron fin stack and where the enclosure for said high-voltage capacitor is terminated by a metal cover electrically connected to the high-voltage pole of said capacitor, with the aim of allowing capacitive coupling between the input end of said thyratron and the output end of said capacitor, and also allowing capacitive coupling between the output end of said thyratron and said dome electrode.

6. The apparatus of claim 1 wherein the metallic fins comprising said multi-fin spark gap thyratron have a flat ring or washer-like profile with a number of small-radius nubs projecting toward the fin's central axis.

7. The apparatus of claim 1 wherein the output of said scalar-longitudinal DC shock waves have a rise time of not more than 800 nanoseconds.

8. The apparatus of claim 1 wherein said scalar-longitudinal DC shock waves have a negative polarity.

9. The apparatus of claim 1 wherein said high-voltage DC power source is a Cockroft-Walton voltage multiplier or a high-voltage DC transformer.

10. The apparatus of claim 1 in which said high-voltage DC power source and thyratron is a Marx bank voltage multiplier.

11. The apparatus of claim 1 in which said target object or person is located within an electrically grounded chamber to receive said scalar-longitudinal DC shock waves.

12. An apparatus transmitting a repeating series of scalar-longitudinal, DC shock waves through a target organism, said apparatus comprising:
a device having an electrical system that comprises:
a high-voltage DC power source electrically connected to
a capacitor that is switchably connected to
a dome electrode via
a multi-fin-spark-gap thyratron;
the electrical system configured to produce a repeating series of scalar-longitudinal DC shock waves of short rise-time; and
the device configured to direct said shock waves toward a target organism, whereby the target organism may experience healing and/or an improvement of health.

13. The apparatus of claim 12 wherein said capacitor, thyratron and dome electrode are together aligned in collinear fashion substantially along a central axis extending perpendicular to the dome electrode.

14. The apparatus of claim 12 wherein said capacitor is electrically connected proximate to the input end of the thyratron and where the output end of the thyratron is electrically connected proximate to the dome electrode and wherein means are provided for varying the length of said electrically connecting means.

15. The apparatus of claim 12 wherein said capacitor is connected to the input end of said thyratron by electrically connective means having a diameter sufficiently large to provide a low-inductance path for said charge being intermittently discharged through said thyratron, and where the output end of said thyratron is connected to the dome electrode by electrically connective means having a diameter sufficiently large to provide a low-inductance path for said charge being intermittently discharged to said dome electrode.

16. The apparatus of claim 12 wherein the enclosure for said thyratron is terminated at each of its ends by metal covers each electrically connected to an outer metallic fin of the thyratron fin stack and where the enclosure for said high-voltage capacitor is terminated by a metal cover electrically connected to the high-voltage pole of said capacitor, with the aim of allowing capacitive coupling between the input end of said thyratron and the output end of said capacitor, and also allowing capacitive coupling between the output end of said thyratron and said dome electrode.

17. The apparatus of claim 12 wherein the metallic fins comprising said multi-fin spark gap thyratron have a flat ring or washer-like profile with a number of small-radius nubs projecting toward the fin's central axis.

18. The apparatus of claim 12 wherein the output of said scalar-longitudinal DC shock waves have a rise time of not more than 800 nanoseconds.

19. The apparatus of claim 12 wherein said scalar-longitudinal DC shock waves have a negative polarity.

20. The apparatus of claim 12 wherein said high-voltage DC power source is a Cockroft-Walton voltage multiplier or a high-voltage DC transformer.

21. The apparatus of claim 12 in which said high-voltage DC power source and thyratron is a Marx bank voltage multiplier.

22. The apparatus of claim 12 in which said target organism is located within an electrically grounded chamber to receive said scalar-longitudinal DC shock waves.

23. A method for transmitting a repeating series of scalar-longitudinal, DC shock waves through a target object or person, said method comprising:
forming a device having an electrical system that comprises:
a high-voltage DC power source electrically connected to
a capacitor that is switchably connected to
a dome electrode via
a multi-fin-spark-gap thyratron;
configuring the electrical system to produce a repeating series of scalar-longitudinal DC shock waves of short rise-time; and
configuring the device to direct said shock waves toward a target object or person, wherein the rate of time flow experienced by the target object or person is slowed.

24. The method of claim 23 wherein said capacitor, thyratron and dome electrode are together aligned in collinear fashion substantially along a central axis extending perpendicular to the dome electrode.

25. The method of claim 23 wherein said capacitor is electrically connected proximate to the input end of the thyratron and where the output end of the thyratron is electrically connected proximate to the dome electrode and wherein means are provided for varying the length of said electrically connecting means.

26. The method of claim 23 wherein said capacitor is connected to the input end of said thyratron by electrically connective means having a diameter sufficiently large to provide a low-inductance path for said charge being intermittently discharged through said thyratron, and where the output end of said thyratron is connected to the dome electrode by electrically connective means having a diameter sufficiently large to provide a low-inductance path for said charge being intermittently discharged to said dome electrode.

27. The method of claim 23 wherein the enclosure for said thyratron is terminated at each of its ends by metal covers each electrically connected to an outer metallic fin of the thyratron fin stack and where the enclosure for said high-voltage capacitor is terminated by a metal cover electrically connected to the high-voltage pole of said capacitor, with the aim of allowing capacitive coupling between the input end of said thyratron and the output end of said capacitor, and also allowing capacitive coupling between the output end of said thyratron and said dome electrode.

28. The method of claim 23 wherein the metallic fins comprising said multi-fin spark gap thyratron have a flat ring or washer-like profile with a number of small-radius nubs projecting toward the fin's central axis.

29. The method of claim 23 wherein the output of said scalar-longitudinal DC shock waves have a rise time of not more than 800 nanoseconds.

30. The method of claim 23 wherein said scalar-longitudinal DC shock waves have a negative polarity.

31. The method of claim 23 wherein said high-voltage DC power source is a Cockroft-Walton voltage multiplier or a high-voltage DC transformer.

32. The method of claim 23 in which said high-voltage DC power source and thyratron is a Marx bank voltage multiplier.

33. The method of claim 23 in which said target object or person is located within an electrically grounded chamber to receive said scalar-longitudinal DC shock waves.

34. A method for transmitting a repeating series of scalar-longitudinal, DC shock waves through a target organism, said method comprising:
forming a device having an electrical system that comprises:
a high-voltage DC power source electrically connected to
a capacitor that is switchably connected to
a dome electrode via
a multi-fin-spark-gap thyratron;
configuring the electrical system to produce a repeating series of scalar-longitudinal DC shock waves of short rise-time; and
configuring the device to direct said shock waves toward a target object, for the purpose of healing and/or improving the health of the organism.

35. The method of claim 34 wherein said capacitor, thyratron and dome electrode are together aligned in collinear fashion substantially along a central axis extending perpendicular to the dome electrode.

36. The method of claim 34 wherein said capacitor is electrically connected proximate to the input end of the thyratron and where the output end of the thyratron is electrically connected proximate to the dome electrode and wherein means are provided for varying the length of said electrically connecting means.

37. The method of claim 34 wherein said capacitor is connected to the input end of said thyratron by electrically connective means having a diameter sufficiently large to provide a low-inductance path for said charge being intermittently discharged through said thyratron, and where the output end of said thyratron is connected to the dome electrode by electrically connective means having a diameter sufficiently large to provide a low-inductance path for said charge being intermittently discharged to said dome electrode.

38. The method of claim 34 wherein the enclosure for said thyratron is terminated at each of its ends by metal covers each electrically connected to an outer metallic fin of the thyratron fin stack and where the enclosure for said high-voltage capacitor is terminated by a metal cover electrically connected to the high-voltage pole of said capacitor, with the aim of allowing capacitive coupling between the input end of said thyratron and the output end of said capacitor, and also allowing capacitive coupling between the output end of said thyratron and said dome electrode.

39. The method of claim 34 wherein the metallic fins comprising said multi-fin spark gap thyratron have a flat ring or washer-like profile with a number of small-radius nubs projecting toward the fin's central axis.

40. The method of claim 34 wherein the output of said scalar-longitudinal DC shock waves have a rise time of not more than 800 nanoseconds.

41. The method of claim 34 wherein said scalar-longitudinal DC shock waves have a negative polarity.

42. The method of claim 34 wherein said high-voltage DC power source is a Cockroft-Walton voltage multiplier or a high-voltage DC transformer.

43. The method of claim 34 in which said high-voltage DC power source and thyratron is a Marx bank voltage multiplier.

44. The method of claim 34 in which said target organism is located within an electrically grounded chamber to receive said scalar-longitudinal DC shock waves.
